(19) United States
(12) Patent Application Publication
(10) Pub. No.: US 2020/0411137 A1
(43) Pub. Date: Dec. 31, 2020

(54) Drug Recommendation Method and System

(71) Applicant: Guangzhou University, Guangzhou (CN)

(72) Inventors: Wenbin Liu, Guangzhou (CN); Peng Xu, Guangzhou (CN); Gang Fang, Guangzhou (CN); Xiangzhen Zan, Guangzhou (CN); Yugai Du, Guangzhou (CN)

(73) Assignee: Guangzhou University, Guangzhou (CN)

(21) Appl. No.: 16/451,045

(22) Filed: Jun. 25, 2019

(51) Int. Cl. G16B 40/00; G16H 70/40; G16H 20/10; G16B 5/00; G16B 50/00

(52) U.S. Cl. CPC G16B 40/00; G16H 70/40; G16B 50/00; G16B 5/00; G16H 20/10

(57) ABSTRACT

The present invention discloses a drug recommendation method and system. The method includes: obtaining a drug information set related to a cancer to be treated and a gene set for an individual to be treated; calculating a treatment level of each drug according to side effect information of each drug and gene information targeted by each drug; selecting a drug of the highest treatment level to be added to the selected drug set; deleting a gene corresponding to the drug of the highest treatment level from the gene set; calculating the number of targeted genes of each drug; determining whether a value greater than a preset number value exists in the number of targeted genes, to obtain a determining result; if not, determining a drug in the selected drug set as a recommended drug; and if yes, returning to the treatment level calculating step. The drug recommendation method and system of the present invention provide recommendation of a corresponding drug for a specific disease.

### TECHNICAL FIELD

The present invention relates to the field of medicines, and in particular, to a drug recommendation method and system.

### BACKGROUND

What is personalized medicine? Ideally, our vision for personalized medicine includes excavating some key cancer features that can be customized to each cancer patient to achieve optimal therapeutic schedules for successfully treating cancers and preventing recurrence.

To achieve personalized medicine, at least two key problems need to be solved. One problem is to excavate sample-based specific cancer markers, and there are many related acquisition methods so far. The other problem is how to use these cancer markers to find drugs to treat cancers. It is possible to apply personalized medicine from theory to actual cancer treatment only by solving the two problems.

The drug achieves cancer treatment by acting on a target. It is necessary to know the relationship between drugs and targets to explore cancer-related drug information. At present, there are many databases that have organized the relationship between drugs and target genes. Although there is certain data support, the relationship between these drugs and target genes is still sparse, and there are many potential relationships that have not been discovered due to technical limitations. Establishing new connections between existing drugs and targets or finding new targets for a given drug are very important to drug discovery. Conventionally, the discovery of the relationship between drugs and target genes is laborious and costly through experiments. Another feasible method is to predict the relationship between drugs and target genes by calculation. In recent years, many methods have been proposed to solve this problem.

The co-occurrence of biological entities in the documents is a simple, comprehensive and popular technique. The association of entities in these documents can be found through co-occurrence. Zhu et al. has proposed a method for excavating the hidden relationship between compounds and genes from the co-occurrence of the documents. Perlman et al. has proposed a computational framework by combining a logistic regression model with the similarity measurement of multiple drugs and the similarity measurement of multiple genes to indicate the interaction between drugs and targets with a final classification score. Cheng et al. ranks the drugs of a particular target gene based on a two-step diffusion model on a binary drug and target gene network to infer the new interaction between drugs and target genes. Wang et al. predicts the new relationship between drugs and target genes by using association, i.e., associated principles and the intuitive interpretation of information flows of drugs and target genes on heterogeneous networks. These methods alleviate the problem that the new relationship between drugs and targets cannot be quickly obtained through experiments to some extent. However, these methods are used to find the relationship between drugs and targets from a broad perspective. In actual use, a specific drug for a certain disease cannot be directly obtained.

### SUMMARY

An objective of the present invention is to provide a drug recommendation method and system for recommending a corresponding drug for a specific disease.

To achieve the foregoing purpose, the present invention provides the following solution.

A drug recommendation method, comprising:

obtaining a drug information set related to a cancer to be treated and a gene set for an individual to be treated;

calculating a treatment level of each drug according to side effect information of each drug in the drug information set and gene information targeted by each drug in the gene set;

selecting a drug of the highest treatment level to be added to the selected drug set;

deleting a gene corresponding to the drug of the highest treatment level from the gene set;

calculating the number of genes in the gene set targeted by each drug in the drug information set, to obtain the number of targeted genes of each drug;

determining whether a value greater than a preset number value exists in the number of targeted genes, to obtain a determining result;

if the determining result indicates that no value greater than a preset number value exists in the number of targeted genes, determining a drug in the selected drug set as a recommended drug; and

if the determining result indicates that a value greater than a preset number value exists in the number of targeted genes, returning to the step of “calculating a treatment level of each drug according to side effect information of each drug in the drug information set and gene information targeted by each drug in the gene set”.

The present invention further discloses a drug recommendation system, comprising:

a drug and gene obtaining module configured to obtain a drug information set related to a cancer to be treated and a gene set for an individual to be treated;

a treatment level calculating module configured to calculate a treatment level of each drug according to side effect information of each drug in the drug information set and gene information targeted by each drug in the gene set;

a drug selecting module configured to select a drug of the highest treatment level to be added to the selected drug set;

a gene deleting module configured to delete a gene corresponding to the drug of the highest treatment level from the gene set;

a target gene number calculating module configured to calculate the number of genes in the gene set targeted by each drug in the drug information set, to obtain the number of targeted genes of each drug;

a determining module configured to determine whether a value greater than a preset number value exists in the number of targeted genes, to obtain a determining result;

a recommending module configured to determine, if the determining result indicates that no value greater than a preset number value exists in the number of targeted genes, a drug in the selected drug set as a recommended drug; and

a returning module configured to return to the treatment level calculating module if the determining result indicates that a value greater than a preset number value exists in the number of targeted genes.

According to specific embodiments provided by the present invention, the present invention discloses the following technical effects: the drug recommendation method and system disclosed by the present invention select recommended drugs from the drugs related to cancers to be treated according to the side effect information of each drug and the number of target genes of each drug, so that the drug can be recommended for a specific cancer disease, that is, the corresponding drug is recommended for a specific disease.

### BRIEF DESCRIPTION OF THE DRAWINGS

To describe the technical solutions in the embodiments of the present invention or in the prior art more clearly, the following briefly introduces the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a method flowchart of an embodiment of a drug recommendation method of the present invention;

FIG. 2 is a schematic diagram showing a specific embodiment of a circulation process of the drug recommendation method of the present invention;

FIG. 3 is a diagram showing the distribution of the number of drugs for lung adenocarcinoma screening in the embodiment of the drug recommendation method of the present invention; and

FIG. 4 is a block diagram showing a system structure of an embodiment of the drug recommendation system of the present invention.

### DETAILED DESCRIPTION

The following clearly and completely describes the technical solutions in the embodiments of the present invention with reference to the accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

The present invention uses drug side effect information, sample-based cancer marker information, relationship information between drugs and target genes, and relationship information between drugs and cancers, and calculates the treatment level of each drug by the number of side effects of the drug and the importance degree of the target gene, thereby achieving a method of recommending a drug candidate set for cancer treatment for each cancer sample.

To make the foregoing objective, features, and advantages of the present invention clearer and more comprehensible, the present invention is further described in detail below with reference to the accompanying drawings and specific embodiments.

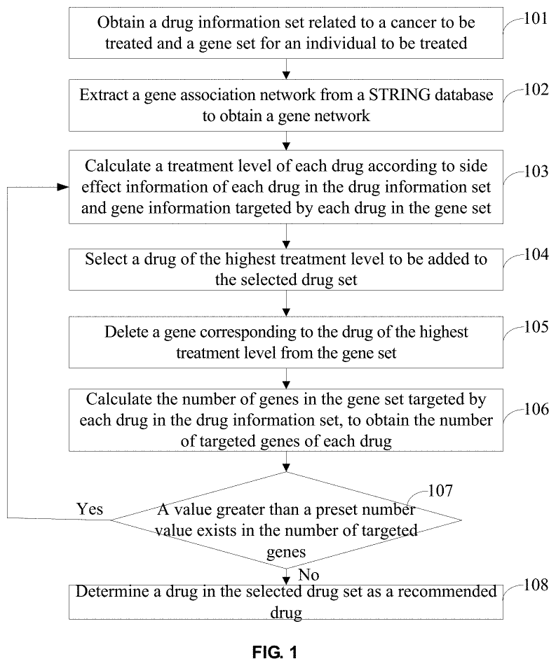

FIG. 1 is a method flowchart of an embodiment of a drug recommendation method of the present invention.

Referring to FIG. 1, the drug recommendation method comprises:

Step 101: a drug information set related to a cancer to be treated and a gene set for an individual to be treated are obtained. The step 101 specifically includes:

a drug related to the cancer to be treated from a corresponding database of cancers and drugs is obtained to obtain a drug set;

a SIDER database is searched for side effect information of each drug in the drug set to obtain a drug information set;

a target gene of each drug in the drug set is obtained to obtain a target gene set;

a gene set in an individual specific marker of the individual to be treated is obtained to obtain a marker gene set; and

an intersection of the target gene set and the marker gene set is taken to obtain a gene set for the individual to be treated.

A specific embodiment of the step 101 is provided below:

a gene expression profile data set of a cancer to be treated is obtained. For example, the cancer to be treated includes, but is not limited to, lung adenocarcinoma (LUAD), primary renal cancer, and endometrial cancer. To eliminate the influence of the condition that some genes are not expressed in multiple samples, the gene expression data is preprocessed before the data is officially used. The preprocessing method includes: deleting genes that are not expressed in more than half of the samples; and filling other unexpressed genes with the mean of the expression quantity of the gene in the expressed sample.

Drug information related to the cancer to be treated is downloaded from ClinicalTrial.gov. 1347 drugs and corresponding side effect information thereof in the SIDER database are obtained, and the obtained drug information set is D={d₁, d₂, . . . , dₘ}.

The gene set T={t₁, t₂, . . . , tₙ} is such that since the relationship between the drug and the target gene is still incomplete, the drugs associated with the cancer to be treated cannot fully cover the genes in these individual specific markers. Therefore, the present embodiment uses the intersection of the target gene set of the drug related to the cancer to be treated and the gene set in the individual specific marker as the drug target gene set T specific for the cancer sample.

Step 102: a gene association network is extracted from a STRING database to obtain a gene network. The STRING database is a currently widely used and well-developed database for which the interaction between genes is searched. A Protein-Protein Interaction (PPI) network is downloaded from the latest version of the STRING database, and 190,692 edges and 10,197 genes included in a high-confidence partial association network with a comprehensive score greater than 900.

Step 103: a treatment level of each drug is calculated according to side effect information of each drug in the drug information set and gene information targeted by each drug in the gene set. The step 103 specifically includes:

the treatment level of each drug is calculated by using a formula

$$S = \frac{{\sum }_{i = 1}^{n} deg ree ( {t}_{i} )}{count ( DS )} ,$$

wherein S represents the treatment level, n represents the number of target genes, tᵢ represents the i-th target gene targeted by a certain drug, degree(tᵢ) represents the degree of the i-th target gene, i.e., the number of genes linked to the i-th target gene in the gene network, DS represents a side effect of a certain drug, and count(DS) represents the number of side effects corresponding to a certain drug; and if there is no side-effect information for a certain drug, a median of the number of side effects of a drug with side effect information is used to represent the number of side effects of a drug without side effect information.

A larger value of S indicates a greater degree drug-targeted genes and smaller side effects. These drugs are more clinically useful and have higher treatment levels.

Step 104: a drug of the highest treatment level is selected to be added to the selected drug set.

Step 105: a gene corresponding to the drug of the highest treatment level is deleted from the gene set, to update the gene set.

Step 106: the number of genes in the gene set targeted by each drug in the drug information set is calculated, to obtain the number of targeted genes of each drug. The gene set in this step is the updated gene set.

Step 107: whether a value greater than a preset number value exists in the number of targeted genes is determined, to obtain a determining result; and

if the determining result indicates that no value greater than a preset number value exists in the number of targeted genes, step 108 is executed.

Step 108: a drug in the selected drug set is determined as a recommended drug; and

if the determining result indicates that a value greater than a preset number value exists in the number of targeted genes, step 103 is executed.

As a preferred embodiment, the preset number value is 5 or 6. The reason for selecting a drug with a target gene number greater than 5 or 6 is that when a drug can only target few genes, the drug is often not effective in practical clinical applications.

A specific embodiment is provided below to explain the circulation process of steps 103-108.

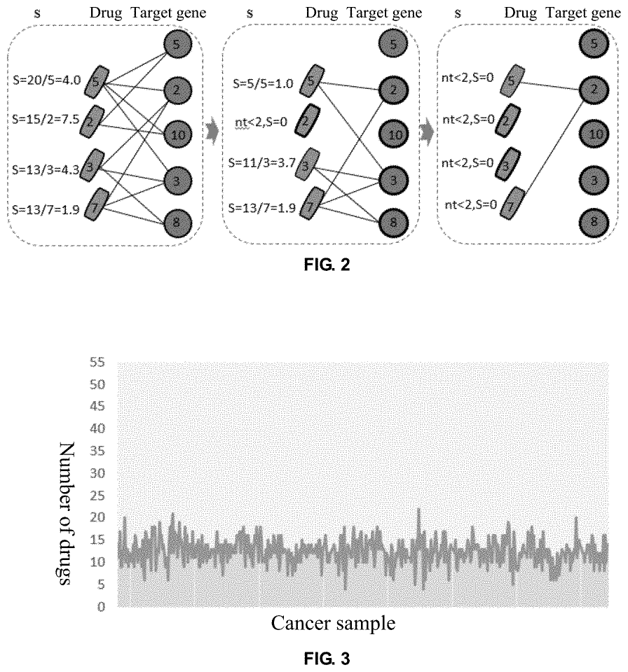

FIG. 2 is a schematic diagram showing a specific embodiment of a circulation process of the drug recommendation method of the present invention.

Referring to FIG. 2, the preset number value in the specific embodiment is 1. The strip-like element represents various drugs, and the figure in the strip-like element represents the number of side effects of each drug. The circle represents a target gene corresponding to the drug, and the figure in the circle represents the size of degree of each target gene. The connecting line between the drug and the gene represents that the drug has a targeted relationship with the gene. The weight S of each drug is calculated by using the number of side effects of the drug and the degree of the target gene, and the drug d₂ with the maximum weight (the second drug, i.e., the drug with two side effects in FIG. 2) is obtained by screening, and all relationships of the genes targeted by the drug d₂ are deleted. In the updated drug-target gene relationship, the weight S of each drug is calculated to obtain a new drug d₃ with the maximum weight (the third drug, i.e., the drug with three side effects in FIG. 2), and in this case, the number of target genes of the remaining drug does not meet the requirements, and the screening is ended. The finally obtained drug list is [d₂, d₃].

FIG. 3 is a diagram showing the distribution of the number of drugs for LUAD screening in the embodiment of the drug recommendation method of the present invention.

Referring to FIG. 3, the LUAD gene expression data set includes 95 normal samples and 514 cancer samples, and 55 LUAD-related drugs are downloaded from ClinicalTrial.gov. According to the method proposed by the present invention, an individual-specific candidate drug set is screened from 55 drugs for each cancer sample to obtain a drug set under each cancer sample. In view of the above, the present invention can recommend different drugs for different samples, and provides drug support for the realization of personalized treatment.

The method of the present invention has the following technical effect.

The present invention calculates the treatment level for each drug of each cancer sample by combining the side effect information of the drug and the information of the drug target gene, and screens drugs according to the treatment level to form a candidate drug set for treating the cancer sample. By means of the method, the clinical application of personalized cancer markers is realized, and more importantly, targeted drug combinations can be provided for different cancer patients, thereby providing a drug basis for personalized treatment for cancer treatment.

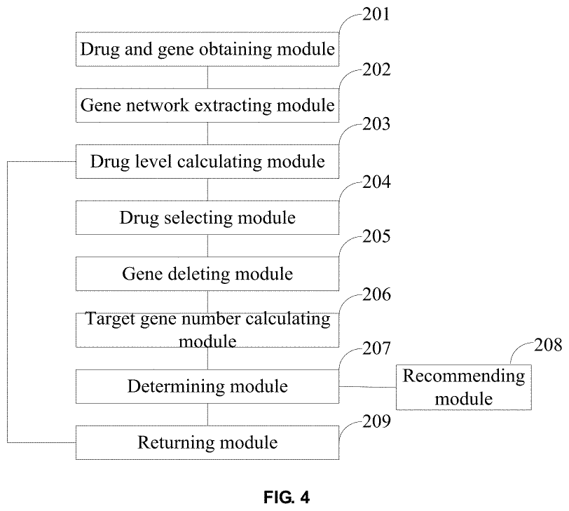

FIG. 4 is a block diagram showing a system structure of an embodiment of the drug recommendation system of the present invention.

Referring to FIG. 4, the drug recommendation system comprises:

a drug and gene obtaining module 201 configured to obtain a drug information set related to a cancer to be treated and a gene set for an individual to be treated;

a gene network extracting module 202 configured to extract a gene association network from a STRING database to obtain a gene network.

a treatment level calculating module 203 configured to calculate a treatment level of each drug according to side effect information of each drug in the drug information set and gene information targeted by each drug in the gene set;

a drug selecting module 204 configured to select a drug of the highest treatment level to be added to the selected drug set;

a gene deleting module 205 configured to delete a gene corresponding to the drug of the highest treatment level from the gene set;

a target gene number calculating module 206 configured to calculate the number of genes in the gene set targeted by each drug in the drug information set, to obtain the number of targeted genes of each drug;

a determining module 207 configured to determine whether a value greater than a preset number value exists in the number of targeted genes, to obtain a determining result;

a recommending module 208 configured to determine, if the determining result indicates that no value greater than a preset number value exists in the number of targeted genes, a drug in the selected drug set as a recommended drug; and

a returning module 209 configured to return to the treatment level calculating module 203 if the determining result indicates that a value greater than a preset number value exists in the number of targeted genes.

As an alternative embodiment, the treatment level calculating module 203 comprises:

a level calculating unit configured to calculate the treatment level of each drug by using a formula

$$S = \frac{{\sum }_{i = 1}^{n} deg ree ( {t}_{i} )}{count ( DS )} ,$$

wherein S represents the treatment level, n represents the number of target genes, tᵢ represents the i-th target gene targeted by a certain drug, degree(tᵢ) represents the degree of the i-th target gene, i.e., the number of genes linked to the i-th target gene in the gene network, DS represents a side effect of a certain drug, and count(DS) represents the number of side effects corresponding to a certain drug; and if there is no side-effect information for a certain drug, use a median of the number of side effects of a drug with side effect information to represent the number of side effects of a drug without side effect information.

As an alternative embodiment, the drug and gene obtaining module 201 comprises:

a drug set obtaining unit configured to obtain a drug related to the cancer to be treated from a corresponding database of cancers and drugs to obtain a drug set;

a side effect searching unit configured to search a SIDER database for side effect information of each drug in the drug set to obtain a drug information set;

a target gene obtaining unit configured to obtain a target gene of each drug in the drug set to obtain a target gene set;

a marker gene obtaining unit configured to obtain a gene set in an individual specific marker of the individual to be treated to obtain a marker gene set; and

an intersection taking unit configured to take an intersection of the target gene set and the marker gene set to obtain a gene set for the individual to be treated.

According to specific embodiments provided by the present invention, the present invention discloses the following technical effects: the drug recommendation method and system disclosed by the present invention select recommended drugs from the drugs related to cancers to be treated according to the side effect information of each drug and the number of target genes of each drug, so that the drug can be recommended for a specific cancer disease, that is, the corresponding drug is recommended for a specific disease.

Several examples are used for illustration of the principles and implementation methods of the present invention. The description of the embodiments is used to help illustrate the method and its core principles of the present invention. In addition, those skilled in the art can make various modifications in terms of specific embodiments and scope of application in accordance with the teachings of the present invention. In conclusion, the content of this specification shall not be construed as a limitation to the invention.

What is claimed is:

1. A drug recommendation method, comprising:
obtaining a drug information set related to a cancer to be treated and a gene set for an individual to be treated;
calculating a treatment level of each drug according to side effect information of each drug in the drug information set and gene information targeted by each drug in the gene set;
selecting a drug of the highest treatment level to be added to the selected drug set;
deleting a gene corresponding to the drug of the highest treatment level from the gene set;
calculating the number of genes in the gene set targeted by each drug in the drug information set, to obtain the number of targeted genes of each drug;
determining whether a value greater than a preset number value exists in the number of targeted genes, to obtain a determining result;
if the determining result indicates that no value greater than a preset number value exists in the number of targeted genes, determining a drug in the selected drug set as a recommended drug; and
if the determining result indicates that a value greater than a preset number value exists in the number of targeted genes, returning to the step of “calculating a treatment level of each drug according to side effect information of each drug in the drug information set and gene information targeted by each drug in the gene set”.

2. The drug recommendation method according to claim 1, wherein the calculating a treatment level of each drug according to side effect information of each drug in the drug information set and gene information targeted by each drug in the gene set specifically comprises:
calculating the treatment level of each drug by using a formula
$$S = \frac{{\sum }_{i = 1}^{n} deg ree ( {t}_{i} )}{count ( DS )} ,$$
wherein S represents the treatment level, n represents the number of target genes, tᵢ represents the i-th target gene targeted by a certain drug, degree(tᵢ) represents the degree of the i-th target gene, i.e., the number of genes linked to the i-th target gene in the gene network, DS represents a side effect of a certain drug, and count(DS) represents the number of side effects corresponding to a certain drug; and if there is no side-effect information for a certain drug, using a median of the number of side effects of a drug with side effect information to represent the number of side effects of a drug without side effect information.

3. The drug recommendation method according to claim 1, wherein the obtaining a drug information set related to a cancer to be treated and a gene set for an individual to be treated specifically comprises:
obtaining a drug related to the cancer to be treated from a corresponding database of cancers and drugs to obtain a drug set;
searching a SIDER database for side effect information of each drug in the drug set to obtain a drug information set;
obtaining a target gene of each drug in the drug set to obtain a target gene set;
obtaining a gene set in an individual specific marker of the individual to be treated to obtain a marker gene set; and
taking an intersection of the target gene set and the marker gene set to obtain a gene set for the individual to be treated.

4. The drug recommendation method according to claim 2, wherein before the calculating a treatment level of each drug according to side effect information of each drug in the drug information set and gene information targeted by each drug in the gene set, the method further comprises:
extracting a gene association network from a STRING database to obtain a gene network.

5. A drug recommendation system, comprising:
a drug and gene obtaining module configured to obtain a drug information set related to a cancer to be treated and a gene set for an individual to be treated;
a treatment level calculating module configured to calculate a treatment level of each drug according to side effect information of each drug in the drug information set and gene information targeted by each drug in the gene set;
a drug selecting module configured to select a drug of the highest treatment level to be added to the selected drug set;
a gene deleting module configured to delete a gene corresponding to the drug of the highest treatment level from the gene set;
a target gene number calculating module configured to calculate the number of genes in the gene set targeted by each drug in the drug information set, to obtain the number of targeted genes of each drug;
a determining module configured to determine whether a value greater than a preset number value exists in the number of targeted genes, to obtain a determining result;
a recommending module configured to determine, if the determining result indicates that no value greater than a preset number value exists in the number of targeted genes, a drug in the selected drug set as a recommended drug; and
a returning module configured to return to the treatment level calculating module if the determining result indicates that a value greater than a preset number value exists in the number of targeted genes.

6. The drug recommendation system according to claim 5, wherein the treatment level calculating module comprises:
a level calculating unit configured to calculate the treatment level of each drug by using a formula
$$S = \frac{{\sum }_{i = 1}^{n} deg ree ( {t}_{i} )}{count ( DS )} ,$$
wherein S represents the treatment level, n represents the number of target genes, tᵢ represents the i-th target gene targeted by a certain drug, degree(tᵢ) represents the degree of the i-th target gene, i.e., the number of genes linked to the i-th target gene in the gene network, DS represents a side effect of a certain drug, and count(DS) represents the number of side effects corresponding to a certain drug; and if there is no side-effect information for a certain drug, use a median of the number of side effects of a drug with side effect information to represent the number of side effects of a drug without side effect information.

7. The drug recommendation system according to claim 5, wherein the drug and gene obtaining module comprises:
a drug set obtaining unit configured to obtain a drug related to the cancer to be treated from a corresponding database of cancers and drugs to obtain a drug set;
a side effect searching unit configured to search a SIDER database for side effect information of each drug in the drug set to obtain a drug information set;
a target gene obtaining unit configured to obtain a target gene of each drug in the drug set to obtain a target gene set;
a marker gene obtaining unit configured to obtain a gene set in an individual specific marker of the individual to be treated to obtain a marker gene set; and
an intersection taking unit configured to take an intersection of the target gene set and the marker gene set to obtain a gene set for the individual to be treated.

8. The drug recommendation system according to claim 6, further comprising:
a gene network extracting module configured to extract a gene association network from a STRING database to obtain a gene network.
